(19) United States
(12) Patent Application Publication
(10) Pub. No.: US 2017/0143536 A1
(43) Pub. Date: May 25, 2017

(54) Medical Device Support System

(71) Applicant: Layna Abreu, New York, NY (US)

(72) Inventor: Layna Abreu, New York, NY (US)

(21) Appl. No.: 14/952,452

(22) Filed: Nov. 25, 2015

(51) Int. Cl. A61F 5/44

(52) U.S. Cl. CPC A61F 5/4408

(57) ABSTRACT

A medical device support system comprises an adjustable waist strap adapted to be worn around the waist of a patient; a first adjustable shoulder strap adapted to be worn across a first shoulder of the patient, a second adjustable shoulder strap adapted to be worn across a second shoulder of the patient, said first and second shoulder straps attached to the waist strap on the front side and the back side of the patient; and a pouch portion attached to the waist strap below the shoulder straps and suspended downwards from the waist strap, said pouch portion having a back layer, a pocket attached to the back layer, an opening at the pocket's upper end for receiving a medical device, and a front layer that covers the pocket and attached to the back layer.

### FIELD OF THE INVENTION

The present invention relates generally to support systems for medical devices. More particularly, the invention relates to a support system for a portable urine collection bag and related tubing.

### BACKGROUND

In cases of certain diseases, trauma, pathological or congenital conditions, or due to surgery, a person's kidney may be rendered partly or wholly non-functional. In these cases, a procedure known as nephrostomy may be performed. The procedure inserts a tube into the urinary tract to divert the flow of the urine from one or both kidneys to and out through the skin. Typically, urine is collected in a pouch or a bag and a patient must support the pouch or bag throughout the day, including when sleeping.

For years, the urine collection bag and tubing has been secured to a patient's body directly on the skin by an adhesive, attached to the clothing by safety pins, or connected to a support pole that moves with the patient. But these options have been less than optimal. Keeping the bag and tubing in direct contact with the patient's skin causes irritation and discomfort. Using a support pole or securing it to the clothing is a physical hindrance, prevents the patient from having a regular life, and leads to lower self-esteem about body image. Harness systems in the form of belts around the patient's waist holding collection bags have been disclosed, for example U.S. Pat. No. 8,226,621 to Timmons and U.S. Pat. No. 5,234,420 to Horton. While these known mechanisms provide some improvement, they offer only limited support because the collection bags are hanging or are attached to the body. They are also uncomfortable to use as the bags move, especially when the patient moves, and limit them from regular everyday activities. Children especially find it difficult to adapt to these bags. Thus, there is a need for an improved support system for removably attaching urine bag with tubing or other such medical devices to patients that is simple, secure, comfortable, and easy to use.

### SUMMARY OF THE INVENTION

The current invention is a support system for holding a medical device such as, a urine collection bag with associated tubing, which may be worn by a patient and optimizes the comfort of and secures the attached medical device. According to one embodiment of the invention, the support system includes a waist strap with attached first and second shoulder straps, and an attached pouch portion housing a pocket for receiving the medical device, the support system adapted to be worn around the waist and across the shoulders of a patient or other user. The shoulder straps provide additional support for carrying the medical device, especially, for example, as the urine collection bags get filled up with urine during the day and become heavier. The design of the pouch allows the pocket carrying the medical device to be concealed from view for improved appearance.

According to one embodiment of the invention, each shoulder strap comprises at least two portions—a first portion and a second portion—that are removably attached to each other to allow adjusting the length of the shoulder strap.

According to another embodiment of the invention, the waist strap has an attached cover to secure the tubing associated with the medical device and keep it in place. According to yet another aspect of the invention, the two shoulder straps are attached on the back side of the patient for better support and stability. The ends of the waist strap and the shoulder straps as well as the pouch layers are secured to each other by Velcro, hook and loop, or other such mechanisms to allow adjustments based on size of the patient.

According to another embodiment of the invention, the support system includes a waist strap with attached shoulder straps, and attached first and second pouch portions housing two pockets for receiving first and second medical devices.

In this way, the support system of the invention provides a structure that is comfortable and resists displacement of the medical device during normal activities like walking and moving. At the same time, the invention allows for easy removal of the medical device. Thus, the invention allows patients, especially children, to wear the medical device in a comfortable, safe, and inconspicuous manner thereby improving patient compliance and patient confidence. These and other aspects and advantages of the invention will be apparent from the following detailed description of the embodiments and the accompanying drawings.

### BRIEF DESCRIPTION OF THE DRAWINGS

FIG. 1 is a front perspective view of a medical device support system on a patient's body, according to one embodiment of the invention.

FIG. 2 is a rear perspective view of a medical device support system on a patient's body, according to one embodiment of the invention.

FIG. 3 is an enlarged top plan view of a waist strap portion of the medical device support system of FIG. 1, according to one embodiment of the invention.

FIG. 4 is an enlarged top plan view of a shoulder straps portion of the medical device support system of FIG. 1, according to one embodiment of the invention.

FIG. 5 is an enlarged top plan view of a pouch portion of the medical device support system of FIG. 1, according to one embodiment of the invention.

FIG. 6 is an enlarged rear perspective view of the medical device support system, according to one embodiment of the invention.

### DETAILED DESCRIPTION OF THE INVENTION

The following detailed description of the invention refers to the accompanying figures.

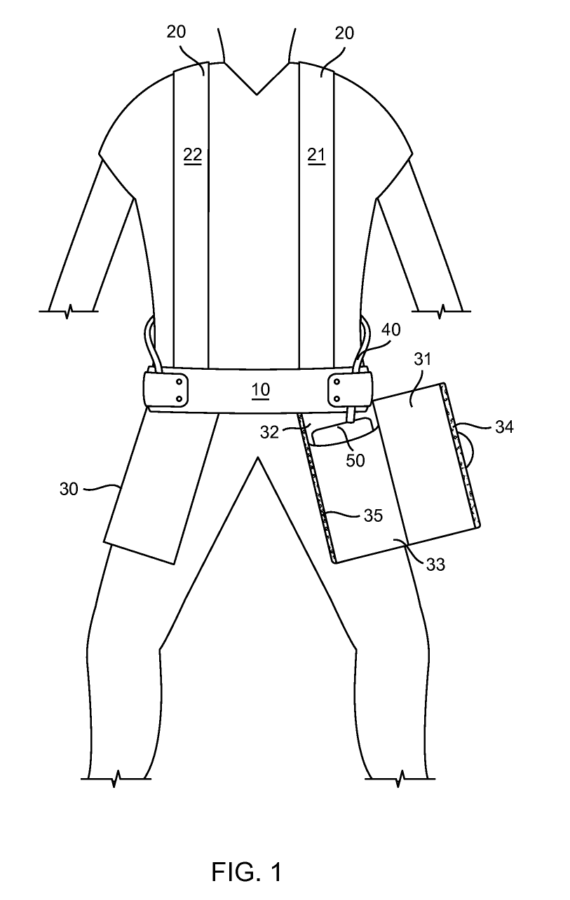

As shown in FIG. 1, a medical device support system 100 in one embodiment comprises a waist portion 10, a shoulder portion 20, and a pouch portion 30. The shoulder portion 20 comprises a first shoulder strap 21 and a second shoulder strap 22. The waist portion and the shoulder portion support the medical device that is housed in the pouch portion. The support system 100 is shown, from a front view, attached as a harness system around a patient's waist and shoulders.

The pouch portion 30 comprises a front layer 31, a back layer 32, and a pocket 33. The pouch 33 holds a medical device 50 such as a urine collection bag. The medical device 50 has associated tubing 40. In nephrostomy patients, for example, one end of the tubing 40 is connected to the kidneys through a patient's skin and the other end is connected to the urine collection bag. The front and back layers of the pouch are removably secured to each other. For example, the front and back layers may each contain Velcro material on at least one side of their edges 34 and 35.

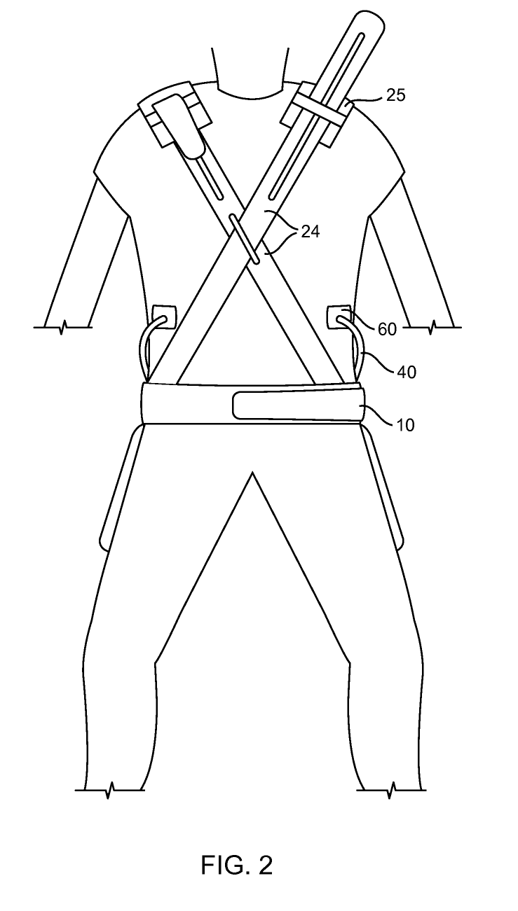

Referring to FIG. 2, the support system 100 is shown, from a rear view, attached as a harness system around the patient's waist and shoulders. The tubing 40 comes out of the patient's skin from an opening 60. Shoulder straps 21 and 22 each comprise a front first piece 23 and a back second piece 24. The first and second pieces of the shoulder strap are secured to each other by a mechanism 25. Mechanism 25 may be any suitable mechanism such as a hook and loop or Velcro. The first and second pieces of the shoulder strap allow the strap to be adjusted based on a patient's size. Alternately, the shoulder straps 21 and 22 may each be a single piece.

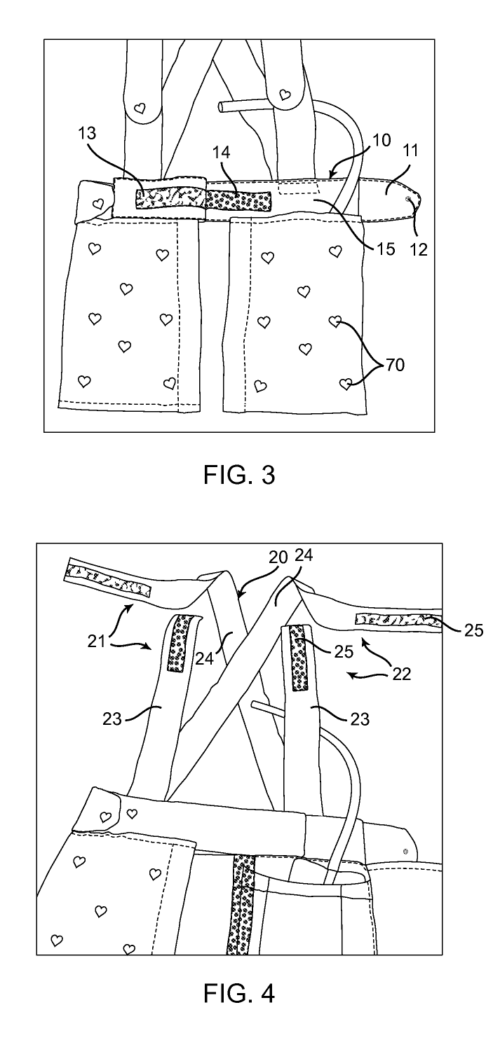

A close-up front view of the waist portion 10 of the support system is shown in FIG. 3. The waist portion 10 comprises a waist strap 15 with opposing ends 13 and 14, and optionally a cover 11. The cover 11 covers the tubing 40 and keeps it secured to the waist strap 15 using a mechanism 12. Mechanism 12 may be, for example, one or more press buttons or hook and loop or Velcro or any other known suitable means. Securing the tubing 40 to the waist strap provides additional support to the tubing, keeps it in place and makes it easier to place the medical device in the pocket 33 by aligning the tubing over the top of the medical device. The opposing ends 13 and 14 of the waist strap allow adjustment of the waist strap according to a patient's size. The ends 13 and 14 are removably secured to each other by any suitable means. For instance, they may be secured by Velcro or hook and loop or buttons.

A close-up front view of the shoulder portion 20 of the support system is shown in FIG. 4. Shoulder strap 21 comprises a first piece 23 in the front and a second piece 24 in the back. Similarly, shoulder strap 22 comprises a first piece 23 and a back piece 24. The first and second pieces are removably secured to each other by any suitable mechanism 25 such as Velcro on the opposing end sides of these pieces. Having the first and second pieces in the shoulder straps allows for additional adjustment to accommodate different sizes of patients. In one embodiment of the invention, each shoulder strap is a single piece.

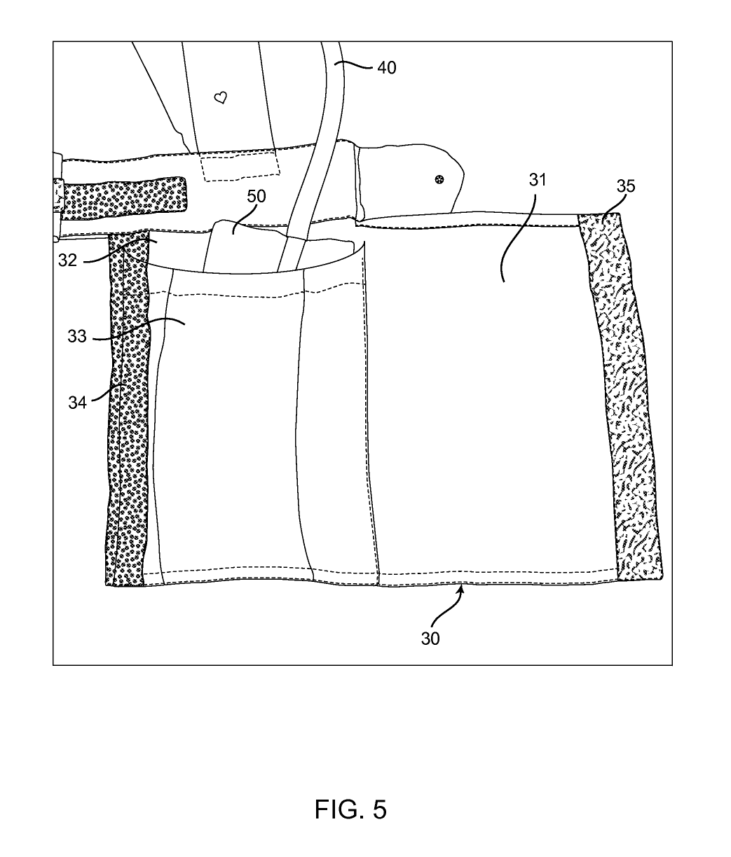

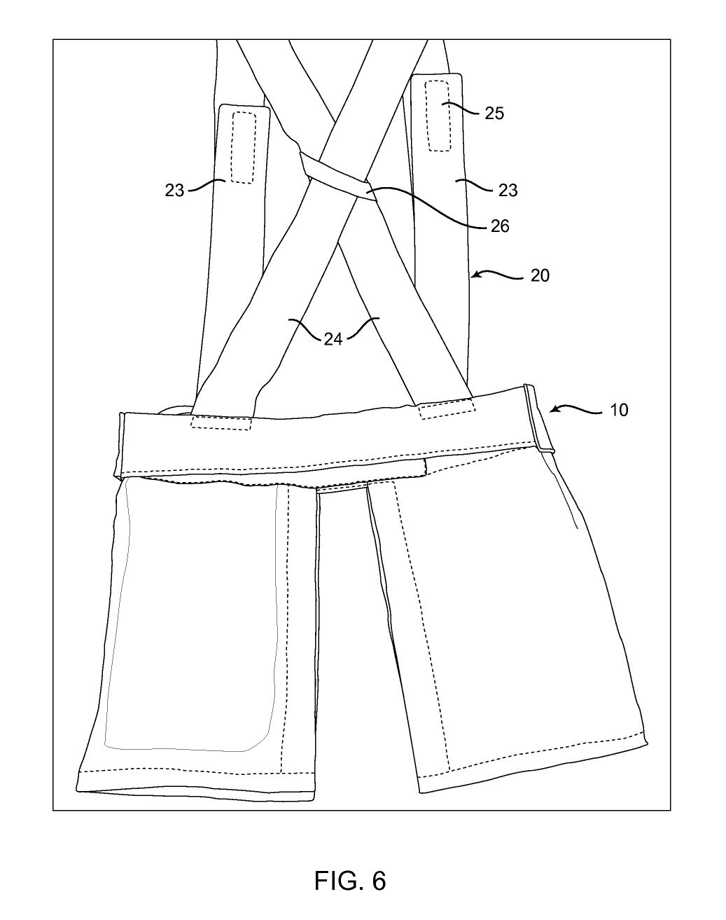

FIG. 5 shows a close-up front view of the pouch portion 30 of the support system. The pouch portion 30 is directly attached to the waist strap 15 and is suspended downwards from the waist strap and below the shoulder strap 21 or 22. The pouch portion is thus supported by both the waist strap and one or both shoulder straps. This provides additional support especially as the medical device such as the urine collection bag fills up with urine as the day progresses and becomes heavier. Heavier bags decrease patient mobility and comfort. The front layer 31 of the pouch portion is attached to the back layer 32 and folds over and covers the pocket 33. Pocket 33 is attached to the back layer along the periphery of the back layer on three sides. The fourth side along the upper end of the pocket forms an opening for receiving the medical device 50. When the front layer 31 folds over the back layer 32, it preferably covers the entire pocket and medical device such that the medical device cannot be seen. Alternately, the front layer 31 does not fold over the back layer 32 but is reinforced as an extra layer over the pocket 33. Or the outer sleeve of the pocket 33 is thick and big enough to not require additional reinforcement or coverage. This increases the confidence of patients, especially children, as they can go about their daily activities without the medical device being seen by others. The pocket 33 provides extra support for the medical device. It keeps the medical device, for example, the urine collection bag, in place as it is snugly held inside the pocket. This greatly increases the patient comfort as the urine bag is not hanging by itself and does not touch the patient's skin. Comfort and mobility are also better because the bag is not attached to the patient's clothing or to a patient's leg making it inconvenient and irritating. The pocket 33 housed between the front and back layers acts as a cushion for the urine bag or other medical device against any injury caused by routine patient movement creating friction between the bag and the patient's skin or clothing. It also allows for easy removal of the bag when it gets filled up.

Referring to FIG. 6, an enlarged rear perspective view of the medical device support system is shown. The second pieces 24 of the shoulder straps 21 and 22 are removably attached to each other on the back side of a patient by any suitable means 26 such as flap, Velcro, loop, button or other known mechanism. In other embodiments of the invention, the second pieces 24 of the shoulder straps are not attached to each other. Alternately, the second pieces 24 may cross each other in an X-shape or be straight or some other known shape. Using two shoulder straps provides extra support for the medical device beyond that provided by the waist strap. The shoulder straps make the support system 100 easier and more secure to wear and keep the medical device in place.

In another embodiment of the invention, the support system 100 has a second pouch portion 30 housing a second medical device 50. The second pouch portion is needed, for example, when patients undergo bilateral nephrostomy and need two urine collection bags. Similar to the first pouch portion 30, the second pouch portion 30 is also attached to the waist strap 15 and is suspended downwards from the waist strap and below the second shoulder strap.

As shown in FIG. 3, the outside of the outer layer 31 of the pouch portion, the outside of the waist strap 15, outside of the cover 11, and/or the outside of the shoulder straps 21 and 22 may be decorated wholly or partially with a decoration 70. The decoration 70 may be any shape, character, pattern or design or add-ons like pretty buttons to cater to patient's taste and preference. The decoration improves the appearance of the support system and increases patient satisfaction and compliance. The support system of the present invention keeps the medical device concealed and can be worn by patients over or under their clothing without feeling embarrassed. The waist portion, the shoulder portion and the pouch portion are made of any known suitable material that will not irritate a patient's skin, for example, cotton, cotton blend, polyester, nylon or others. The material used is preferably lightweight, breathable, easy to wipe and clean, and absorbent. The pouch and pocket may be manufactured in various sizes to accommodate various sizes of the medical devices. The waist portion, the shoulder portion, and the pouch portion may all be made out of a single piece of material or one or more portions may be made of separate pieces of material and then attached.

It is to be understood that the above described arrangements are only illustrative of the application of the principles of the present invention. Numerous modifications and alternative arrangements and applications may be devised by those skilled in the art without departing from the spirit and scope of the present invention and the following claims are intended to cover such modifications and arrangements.

What is claimed is:

1. A medical device support system comprising:
an adjustable waist strap adapted to be worn around the waist of a patient; said adjustable waist strap having opposed first and second ends and a central portion extending between the opposed ends;
a first adjustable shoulder strap having opposed first and second ends and adapted to be worn across a first shoulder of the patient, said opposed ends of the first adjustable shoulder strap attached to the waist strap on a front side and a back side of the patient;
a second adjustable shoulder strap having opposed first and second ends and adapted to be worn across a second shoulder of the patient, said opposed ends of the second adjustable shoulder strap attached to the waist strap on the front side and the back side of the patient; and
a pouch portion attached to a front side of the waist strap below the shoulder straps and suspended downwards from the waist strap, said pouch portion having a back layer, a pocket attached to the back layer along the periphery of the back layer, an opening at the pocket's upper end for receiving a medical device, and a front layer that covers the pocket and attached to the back layer.

2. The medical device support system of claim 1, wherein the adjustable waist strap further comprises a cover portion attached to the waist strap that folds over and secures a tubing to the waist strap, said tubing attached to the medical device on one end.

3. The medical device support system of claim 1, wherein the first end of the waist strap and the second end of the waist strap are removably secured to each other and adjust the length of the waist strap; and
the back layer and the front layer of the pouch portion are removably secured to each other.

4. The medical device support system of claim 3, wherein the ends of the waist strap, and the back and front layers of the pouch portion are removably secured to each other by Velcro.

5. The medical device support system of claim 1, wherein
the first adjustable shoulder strap comprises a first piece and a second piece, the first piece and the second piece removably secured to each other and adjust the length of the first shoulder strap; and
the second adjustable shoulder strap comprises a first piece and a second piece, the first piece and the second piece removably secured to each other and adjust the length of the second shoulder strap.

6. The medical device support system of claim 5, wherein the first and second pieces of the adjustable shoulder straps are removably secured to each other by Velcro.

7. The medical device support system of claim 1, wherein the first adjustable shoulder strap and the second adjustable shoulder strap are secured to each other on the back side of the patient.

8. The medical device support system of claim 1, wherein the medical device is a urine collection bag.

9. A medical device support system comprising:
an adjustable waist strap adapted to be worn around the waist of a patient; said adjustable waist strap having opposed first and second ends and a central portion extending between the opposed ends;
a first adjustable shoulder strap having opposed first and second ends and adapted to be worn across a first shoulder of the patient, said opposed ends of the first adjustable shoulder strap attached to the waist strap on a front side and a back side of the patient;
a second adjustable shoulder strap having opposed first and second ends and adapted to be worn across a second shoulder of the patient, said opposed ends of the second adjustable shoulder strap attached to the waist strap on the front side and the back side of the patient;
a first pouch portion attached to a front side of the waist strap below the first shoulder strap and suspended downwards from the waist strap, said pouch portion having a back layer, a first pocket attached to the back layer along the periphery of the back layer, an opening at the pocket's upper end for receiving a first medical device, and a front layer that covers the first pocket and attached to the back layer; and
a second pouch portion attached to the front side of the waist strap below the second shoulder strap and suspended downwards from the waist strap, said pouch portion having a back layer, a second pocket attached to the back layer along the periphery of the back layer, an opening at the pocket's upper end for receiving a second medical device, and a front layer that covers the second pocket and attached to the back layer.

10. The medical device support system of claim 9, wherein the medical devices are urine collection bags.

11. The medical device support system of claim 9, wherein
the first end of the waist strap and the second end of the waist strap are removably secured to each other and adjust the length of the waist strap; and
the back layer and the front layer of the first and second pouch portions are removably secured to each other.

12. The medical device support system of claim 9, wherein
the first adjustable shoulder strap comprises a first piece and a second piece, the first piece and the second piece removably secured to each other and adjust the length of the first shoulder strap; and
the second adjustable shoulder strap comprises a first piece and a second piece, the first piece and the second piece removably secured to each other and adjust the length of the second shoulder strap.

13. The medical device support system of claim 9, wherein the adjustable waist strap further comprises:
a first cover portion attached to the waist strap that folds over and secures a first tubing to the waist strap, said first tubing attached to the first medical device on one end; and
a second cover portion attached to the waist strap that folds over and secures a second tubing to the waist strap, said second tubing attached to the second medical device on one end.
